(19) United States
(12) Patent Application Publication
(10) Pub. No.: US 2005/0152993 A1
(43) Pub. Date: Jul. 14, 2005

(54) Composition for and method of treatment for skin ailments

(76) Inventor: Mariana De Oliveira, Ontario (CA)

Correspondence Address: MILLER THOMPSON, LLP, 20 QUEEN STREET WEST, SUITE 2500, TORONTO, ON, M5H 3S1, CA

(21) Appl. No.: 10/502,321

(22) PCT Filed: Jan. 25, 2002
(86) PCT No.: PCT/CA02/00090

(51) Int. Cl. A61K 35/78; A61K 33/36; A61K 33/04

(52) U.S. Cl. 424/669; 424/725.1; 424/742; 424/750; 424/745; 424/725; 424/711

(57) ABSTRACT

Improved compositions for treating a wide variety of skin ailments, including a body wash composition, spray composition and cream composition and the use of these novel compositions in a system or method of treating a wide variety of skin ailments containing a herbal extract selected from mallow extract, rosemary extract and eucalyptus extract.

### FIELD OF THE INVENTION

This invention relates in general to a treatment for a wide variety of skin aliments and more particularly to a medication for treating this wide variety of skin ailments having three different formulations, a system for using the medication, and a method of medical treatment.

### BACKGROUND OF THE INVENTION

There are a wide variety of skin ailments that vary in their seriousness, but all in some way affect the outer layer of the skin or epidermis and sometimes beneath the outer layer of epidermis. This wide variety of skin ailments includes but is not limited to: bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot fungus, boils, chapped hands and lips, cuts and scrapes and herpes simplex.

Some of these skin ailments are commonly understood such as bruises, scars, muscular joints and aches, rashes, insect bites, dry itchy scaly skin, skin discoloration, blisters, burns, bee stings, boils, chapped hands and lips, cuts and scrapes.

Cold sores are inflamed blisters usually in or near the mouth and are caused by a herpes simplex infection. Eczema is defined as a non-infective superficial inflammation of the skin, usually with itching and vesicular discharge. Rosacea is a condition in which certain facial blood vessels enlarge thereby giving the cheeks and nose a flush appearance. Shingles is a disease caused by the herpes virus and is characterized by a rash of minute blisters on the skin. Moles are raised patches of skin made dark by an abnormally high concentration of melanin. Acne may be more clinically defined as a severe seborrhoea complicated by pustule formation in the hair follicles. Psoriasis is a skin disorder that includes the presence of small elevations of skin that may be characterized as elevated red lesions, plaques or pustules on the skin, which eventually result in silvery scales. Athlete's foot is a ringworm infection of the foot. Herpes simplex is a herpesvirus that usually causes localized inflammation.

Thus a composition, system and method of treatment for these wide variety of skin ailments which allows for the successful treatment and relief of symptoms is desirable.

### SUMMARY OF THE INVENTION

An object of one aspect of the present invention is to provide improved compositions or pharmaceutical compositions for use in the treatment of sksin ailments, namely bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex. A further object of the invention is to provide an improved system for treatment of skin ailments or method of medical treatment using novel compositions.

In accordance with one aspect of the present invention there is provided a composition for use in the treatment of skin ailments selected from the group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex comprising of an active agent wherein the active agents are herbal extracts including mallow extract and/or coal tar that may be mixed with a carrier mixture for topical administration.

In accordance with other aspects of the invention there are provided compositions for treating skin ailments namely bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, that include a body wash composition, a spray composition and a cream composition. Conveniently the active agents may include a variety of herbal extracts, including mallow extract and/or coal tar.

In accordance with another aspect of the invention there is provided a system for use in skin ailment treatment that includes using a body wash composition, a spray composition and a cream composition wherein the active agents include, herbal extracts including mallow extract and/or coal tar solution in therapeutic effective doses and a carrier mixture for topical application.

In a final aspect of the present invention there is provided a method of treating skin ailments by topically administering to a human having skin ailments, an effective amount of herbal extracts, including mallow extract and/or coal tar wherein the herbal extracts are present in a topically acceptable pharmaceutical body wash composition, spray composition and cream composition.

### DETAILED DESCRIPTION OF THE PREFERRED EMBODIMENT

The present invention relates to novel compositions and method of applying these compositions, which comprise of a selected mixture of herbal extracts and/or coal tar, which have the ability to reduce inflammation and other symptoms associated with skin ailments namely, bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, thereby providing relief from the associated symptoms and encouraging healthy skin growth. The topical application of these novel compositions acts to treat skin, which has these ailments. Topical application means the introduction of the novel compositions to the skin via a suitable base or vehicle. Conventional understanding of topical applications include but are not limited to creams, ointments, lotions, gels, pastes, powders, sprays, washes and the like.

The first novel composition of the present invention is comprised of the following essential herbal extracts and/or coal tar which have been formulated into a body wash composition, which has demonstrated in a reduction of inflammation associated with the disclosed skin ailments. Each of the components herein described is believed to contribute to the natural healing process of the afflicted area although the exact mechanisms are not completely understood. The essential components of the novel body wash composition are: mallow extract, rosemary oil, eucalyptus oil, a blend of proteins and wheat germ oil and/or coal tar. The essential components are included in a suitable carrier for a body wash. More particularly, a preferred composition of the body wash can contain a mixture of components that act as a carrier, the herbal extracts and/or coal tar in the following proportions given in weight percents as indicated in Table 1.

TABLE 1
Skin Ailment Body Wash

| | Percentage | |
|---|---|---|
| Substance | Range | Function |
| Water | 44.19-49.12 | Inert ingredient |
| Sodium Laureth Sulfate | 31.5-38.5 | Emulsifiers |
| Cocamide Dea | 4.5-5.5 | Thicker and foaming agent. |
| | | Assists in removing fatty |
| | | acids |
| Cocamidopropyl Betaine | 0.9-1.1 | Surfactant used as a |
| | | cleanser. |
| Propylene Glycol | 0.27-0.33 | Cleanser |
| Citric Acid | 0.27-0.33 | PH Adjuster |
| Coal Tar Topical Solution | 0.10-6.6 | Active |
| Sodium Chloride | 1.8-2.2 | Thicker |
| Sodium Layroyl Sarcosinate | 1.8-2.2 | Cleansing |
| Wheat Germ Oil | 0.45-0.55 | Active |
| Blends of Proteins | 0.18-0.22 | Active |
| Methylchloroisothiazolinone | 0.045-0.055 | Preservative |
| Mallow Extract | 0.9-1.1 | Active |
| Rosemary Oil | 0.45-0.55 | Active |
| Eucalyptus Oil | 0.45-0.55 | Active/Counterirritant |
| Carbomer | 0.9-1.1 | Gelling Agent |

It will be apparent from the foregoing that the total percentage weight adds up to 100% and therefore the water component of the Skin Ailment Body Wash will make up the remaining percentage after adding up the percentages of the remaining components.

Eucalyptus has antiseptic, cooling and stimulating properties. Rosemary is very rich in natural mineral and has soothing, cleansing, stimulating and astringent properties. Mallow has stimulating soothing properties associated with inflammation.

Coal tar preparations have been approved for treating a variety of skin ailments and problems such as dandruff and psoriasis. Coal tar is a byproduct of treated bituminous coal. Although long term exposure (20-25 years) to high concentrations of coal tar has been associated with skin cancer, topical compositions that are not used for long periods of time are considered fairly safe as the products are in contact with the skin for short periods of time. Furthermore, trace amounts of coal tar are not considered injurious to the skin.

A second novel composition of the present invention is comprised of the following essential herbal extracts alone which have been formulated into a body spray composition, which has demonstrated in a reduction of symptoms including inflammation associated with the identified skin ailments. Each of the components herein described is believed to contribute to the natural healing process of the afflicted area although the exact mechanisms are not completely understood. The essential components of the novel body spray composition are: mallow extract, rosemary oil, and eucalyptus oil. More particularly a preferred composition of the body spray can contain a mixture of components that act as the carrier, herbal extracts in the following proportions given in weight percents as indicated in Table 2.

TABLE 2
Skin Ailment Spray

| Substance | Percentage Range | Function |
|---|---|---|
| Water | 21.573-26.367 | Inert |
| Alcohol | 63-77 | Inert |
| Sodium Iodide | 2.7-3.3 | Carrier |
| Potassium Iodide | 1.35-1.65 | Carrier |
| Sodium Thiosulphate | 0.027-0.033 | Neutralizer |
| Mallow Solution | 0.9-1.1 | Active |
| Rosemary Solution | 0.225-0.27 | Active |
| Eucalyptus Oil | 0.225-0.27 | Active |

A third novel composition of the present invention is comprised of the following essential herbal extracts and/or coal tar which have been formulated into a cream composition, which has demonstrated a reduction of associated symptoms including inflammation associated with the identified skin ailments. Each of the components herein described is believed to contribute to the natural healing process of the afflicted area although the exact mechanisms are not completely understood. The essential components of the novel cream composition are: mallow extract, chamomile extract, tea tree solution and/or coal tar solution. More particularly a preferred composition of the body cream can contain a mixture of components that act as a carrier, herbal extracts and/or coal tar solution in the following proportions given in weight percents as indicated in Table 3.

TABLE 3
Skin Ailment Cream

| Substance | Percentage Range | Function |
|---|---|---|
| Water | 52.10-57.4695 | Inert ingredient |
| Propylene Glycol | 3.6-4.4 | Cleanser |
| Mineral Oil | 2.7-3.3 | Oil Base |
| Cetyl Alcohol | 3.6-4.4 | Emulsifier |
| Stearic Acid | 3.6-4.4 | Emulsifier |
| Glycerol Monostearate | 2.7-3.3 | Emulsifier |
| F D & C Yellow #5 Solution | 0.045-0.055 | Colour |
| D & C Red #33 Solution | 0.0045-0.055 | Colour |
| Chamomile Extract | 0.9-1.1 | Active - Mineral |
| Carbomer | 3.6-4.4 | Gelling Agent |
| Methyl Paraben | 0.225-0.275 | Preservative |
| Propyl Paraben | 0.09-0.11 | Preservative |
| Triethanolamine | 0.54-0.66 | Emulsifier |
| Milk Amino Acids | 2.25-2.75 | Active |
| Tea Tree Solution | 2.7-3.3 | Active |
| Mallow Solution | 0.9-1.1 | Active |
| Coal Tar | 0.10-6.325 | Active |
| Dimethicone | 0.45-0.55 | Luster Agent |
| Glycerine | 2.7-3.3 | Humectant |
| Petrolatum | 0.9-1.1 | Emollient |
| Cetearyl Alcohol | 2.25-2.75 | Emollient/Thicker |
| Ceteareth 20 | 2.25-2.75 | Lubricant |
| Polysorbate 20 | 1.8-2.2 | Emulsifier |

The novel compositions may be used together as a system for use in the treatment of skin ailments, namely bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex. More specifically, in operation, the user may apply the body wash composition to the area of skin that is affected with the ailment. The body wash composition may be gently massaged into the affected area then rinsed with warm water and dried thoroughly. The novel body spray composition may be applied to the affected area and allowed to dry. Once the body spray has dried, the novel body cream may be applied to the affected area by massaging the body cream into the area. The system may be used on the affected area two to three times a day.

The novel compositions may each be used separately or in combination as a method of treating the identified skin ailments, comprising of topically administering to a human having the skin ailment, the novel body wash composition, the body spray composition or the body cream composition or a combination of the novel compositions.

The following examples further illustrate various aspects of the invention.

### EXAMPLE 1

In one particular example favourable results against the skin ailment, shingles were observed by utilizing a Skin Ailment Body Wash, a Skin Ailment Spray and a Skin Ailment Cream bearing the following specific percentage by weight (the specific numerical percentages relate to the specific components described earlier, for example, the second item 35.00 under Body Wash relates to 35% by volume of Sodium Laureth Sulfate, while the second item under Body Spray relates to 70% by weight alcohol and the second item under Body Cream relates to 4% by weight propylene glycol and so on) namely:

| Body Wash | | Body Spray | | Body Cream | |
|---|---|---|---|---|---|
| 44.65 | Water | 23.97 | Water | 52.245 | Water |
| 35.00 | Sodium Laureth Sulfate | 70.00 | Alcohol | 4.00 | Propylene Glycol |
| 5.00 | Cocamide Dea | 3.00 | Sodium Iodide | 3.00 | Mineral Oil |
| 1.00 | Cocamidopropyl Betaine | 1.50 | Potassium | 4.00 | Cetyl Alcohol |
| | | | Iodide | | |
| 0.30 | Propylene Glycol | 0.03 | Sodium | 4.00 | Stearic Acid |
| | | | Thiosulphate | | |
| 0.30 | Citric Acid | 1.00 | Mallow | 3.00 | Glycerol Monostearate |
| | | | Solution | | |
| 6.00 | Coal Tar Topical Solution | 0.25 | Rosemary | 0.05 | F D & C Yellow #5 |
| | | | Solution | | Solution |
| 2.00 | Sodium Chloride | 0.25 | Eucalyptus Oil | 0.005 | D & C Red #33 |
| | | | | | Solution |
| 2.00 | Sodium Layroyl | | | 1.00 | Chamomile Extract |
| | Sarcosinate | | | | |
| 0.05 | Wheat Germ Oil | | | 4.00 | Carbomer |
| 0.20 | Blends of Proteins | | | 0.25 | Methyl Paraben |
| 0.05 | Methylchloroisothiazolinone | | | 0.10 | Propyl Paraben |
| 1.00 | Mallow Extract | | | 0.60 | Triethanolamine |
| 0.5 | Rosemary Oil | | | 2.50 | Milk Amino Acids |
| 0.5 | Eucalyptus Oil | | | 3.00 | Tea Tree Solution |
| 1.00 | Carbomer | | | 1.00 | Mallow Solution |
| | | | | 5.75 | Coal Tar |
| | | | | 0.50 | Dimethicone |
| | | | | 3.00 | Glycerine |
| | | | | 1.00 | Petrolatum |
| | | | | 2.50 | Cetearyl Alcohol |
| | | | | 2.50 | Ceteareth 20 |
| | | | | 2.00 | Polysorbate 20 |

More particularly, by following the steps of:
(a) apply the body wash composition to the area of skin effected with the ailment, and gently massaging same to the effected area for a time period of up to ten minutes and then rinsing same with warm water and then drying same thereof;
(b) then apply the body spray of the specific composition referred to above to the effected area, and allowing same to dry;
(c) then apply the body cream having the specific composition referred to above; and
(d) repeating the steps two to three times a day.

By following the procedure referred to above, one observed a marked improvement over a span of one month where the area of skin showed at least 50% reduction of the shingles. In some instances the shingles completely disappeared.

### EXAMPLE 2

A 45 year old female patient with long standing psoriasis commenced treating her psoriasis by first applying the bodywash composition to the area of skin affected by psoriasis. The bodywash composition was gently massaged into the affected area and then rinsed with warm water and dried thoroughly. The body spray composition was sprayed over the affected area and allowed to dry. Finally, the body cream was massaged into the affected area. The patient repeated this procedure in the morning and the evening over a period of 3-4 weeks. The patient's psoriasis cleared up almost completely, at the end of the four-week period, with only small amounts of psoriasis periodically exhibiting on the patient's skin.

In summary, novel compositions for use in the treatment of skin ailments namely, bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, and corresponding system and method are provided for, wherein the active agents are a combination of herbal extracts including mallow and/or coal tar.

Other variations and modifications of the invention are possible. All such modifications or variations are believed to be within the sphere and scope of the invention as defined by the claims appended hereto.

1. A composition for use in skin ailment treatments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising: active agents and a carrier mixture, wherein said active agents are herbal extracts in a therapeutic effective dose, wherein said herbal extract is selected from a group consisting of mallow extract, rosemary extract and eucalyptus extract and said active agent mixes with said carrier mixture for administration of said composition.

2. A composition for treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, consisting of the following ingredients in the following percentage range by weight:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(I) Methylchloroisothiazolinone in the range of 0.045-0.045
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

3. A composition for treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex consisting of the following ingredients in the following percentage range by weight:
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

4. A composition for treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex consisting of the following ingredients in the following percentage range by weight:
(a) Water in the range of 47.0205-57.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) F D and C Yellow #5 solution in the range of 0.045-0.055
(h) D and C red #33 solution in the range of 0.0045-0.055
(i) Chamomile extract in the range of 0.9-1.1
(j) Carbomer in the range of 3.6-4.4
(k) Methyl paraben in the range of 0.225-0.275
(l) Propyl paraben in the range of 0.09-0.11
(m) Triethanolamine in the range of 0.54-0.66
(n) Milk amino acids in the range of 2.25-2.75
(o) Tea tree solution in the range of 2.7-3.3
(p) Mallow extract in the range of 0.9-1.1
(q) Coal tar topical solution in the range of 5.175-6.325
(r) Dimethicone in the range of 0.45-0.55
(s) Glycerine in the range of 2.7-3.3
(t) Petrolatum in the range of 0.9-1.1
(u) Cetearyl alcohol in the range of 2.25-2.75
(v) Ceteareth 20 in the range of 2.25-2.75
(w) Polysorbate 20 in the range of 1.8-2.2.

5. A pharmaceutical composition for use in the treatment of skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising a skin ailment treatment agent consisting of at least 5% herbal extracts in an effective skin ailment treatment percentage mixed with a carrier mixture for topical administration to a human.

6. A pharmaceutical composition for use in the treatment of skin ailments as claimed in claim 5 wherein said herbal extract is least one percent mallow extract.

7. A system for use in skin ailment treatments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising a body wash composition, a spray composition and a cream composition, wherein said body wash composition, said spray composition and said cream composition further comprises an active agent and carrier mixture, wherein said active agents are herbal extracts in a therapeutic effective dose, and said active agent mixes with said carrier mixture for administration of said body wash composition, said spray composition and said cream composition.

8. A system for use in skin ailment treatments as claimed in claim 7 wherein said body wash composition consisting of the following ingredients in the following range of proportions based on weight:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(l) Methylchloroisothiazolinone in the range of 0.045-0.055
(m) Mallow extract in the range of 0.9-1.1
(n) Rosemary oil in the range of 0.45-5.5
(o) Eucalyptus oil in the range of 0.45-5.5
(p) Carbomer in the range of 0.9-1.1.

9. A system for use in skin ailment treatments as claimed in claim 7 wherein said spray composition consisting of the following ingredients in the following range of proportions based on weight:
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

10. A system for use in skin ailment treatments as claimed in claim 7 wherein said cream composition consisting of the following ingredients in the following range of proportions:
(a) Water in the range of 47.0205-27.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) Coal tar topical solution in the range of 5.175-6.325
(h) Chamomile extract in the range of 0.9-1.1
(i) Methyl paraben in the range of 0.225-0.275
(j) Propyl paraben in the range of 0.09-0.11
(k) Triethanolamine in the range of 0.54-0.66
(l) Milk amino acids in the range of 2.25-2.75
(m) Mallow extract in the range of 0.9-1.1
(n) Tea tree solution in the range of 2.7-3.3
(o) Dimethicone in the range of 0.45-0.55
(p) Carbomer in the range of 3.6-4.4
(q) Glycerine in the range of 2.7-3.3
(r) Petrolatum in the range of 0.9-1.1
(s) Cetearyl alcohol in the range of 2.25-2.75
(t) Ceteareth 20 in the range of 2.25-2.75
(u) Polysorbate 20 in the range of 1.8-2.2
(v) F D and C Yellow #5 solution in the range of 0.045-0.055
(w) D and C red #33 solution in the range of 0.0045-0.055.

11. A method of treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising topically administering to a human having said skin ailment at least 1% herbal extracts, said herbal extracts being present in a topically acceptable pharmaceutical body wash composition.

12. A method as claimed in claim 11 wherein said topically acceptable pharmaceutical body wash composition consisting of the following ingredients in the following range of proportions:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(l) Methylchloroisothiazolinone in the range of 0.045-0.055
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

13. A method of treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising topically administering to a human having said skin ailment, at least 1% herbal extracts, said herbal extracts being present in a topically acceptable pharmaceutical spray composition.

14. A method of treating skin ailments as claimed in claim 13 wherein said topically acceptable pharmaceutical spray composition consisting of the following ingredients in the following range of proportions:
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

15. A method of treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising topically administering to a human having said skin ailments at least 1% herbal extracts, said herbal extracts being present in a topically acceptable pharmaceutical cream composition.

16. A method of treating skin ailments as claimed in claim 15 wherein said topically acceptable pharmaceutical cream composition consisting of the following ingredients in the following range of proportions:
(a) Water in the range of 47.0205-27.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol monostearate in the range of 2.7-3.3
(g) Coal tar topical solution in the range of 5.175-6.325
(h) Chamomile extract in the range of 0.9-1.1
(i) Methyl paraben in the range of 0.225-0.275
(j) Propyl paraben in the range of 0.09-0.11
(k) Triethanolamine in the range of 0.54-0.66
(j) Milk amino acids in the range of 2.25-2.75
(m) Mallow extract in the range of 0.9-1.1
(n) Tea tree solution in the range of 2.7-3.3
(o) Dimethicone in the range of 0.45-0.55
(p) Carbomer in the range of 3.6-4.4
(q) Glycerine in the range of 2.7-3.3
(r) Petrolatum in the range of 0.9-1.1
(s) Cetearyl alcohol in the range of 2.25-2.75
(t) Ceteareth 20 in the range of 2.25-2.75
(u) Polysorbate 20 in the range of 1.8-2.2
(v) F D and C Yellow #5 solution in the range of 0.045-0.055
(w) D and C red #33 solution in the range of 0.0045-0.055.

17. A method for treating skin ailments selected from a group consisting of bruises, scars, cold sores, eczema, rosacea, shingles, muscular joints and aches, moles, acne, rashes, insect bites, psoriasis, dry itchy scaly skin, skin discoloration, blisters, burns, bee stings, athletes foot, boils, chapped hands and lips, cuts and scrapes and herpes simplex, comprising topically administering to a human having said skin ailments at least 1% herbal extracts, said herbal extracts being present in a topically acceptable pharmaceutical body wash composition, spray composition and cream composition.

18. A method for treating skin ailments as claimed in claim 17 wherein said body wash composition consisting of the following ingredients in the following range of proportions:
(a) Water in the range of 40.19-49.12
(b) Sodium laureth sulphate in the range of 31.5-38.5
(c) Cocamide DEA in the range of 4.5-5.5
(d) Cocamidopropyl betaine in the range of 0.9-1.1
(e) Proylene glycol in the range of 0.27-0.33
(f) Citric acid in the range of 0.27-0.33
(g) Coal tar topical solution in the range of 5.4-6.6
(h) Sodium chloride in the range of 1.8-2.2
(i) Sodium layroyl sarcosinate in the range of 1.8-2.2
(j) Wheat germ oil in the range of 0.45-0.55
(k) Proteins (blends) in the range of 0.18-0.22
(l) Methylchloroisothiazolinone in the range of 0.045-0.055
(m) Mallow extract in the range of 0.9-1.1
(n) Rosemary oil in the range of 0.45-0.55
(o) Eucalyptus oil in the range of 0.45-0.55
(p) Carbomer in the range of 0.9-1.1.

19. A method for treating skin ailments as claimed in claim 18 wherein said spray composition consisting of the following ingredients in the following range of proportions:
(a) Water in the range of 21.573-26.367
(b) Alcohol in the range of 63-77
(c) Sodium Iodide in the range of 2.7-3.3
(d) Potassium iodide in the range of 1.35-1.65
(e) Sodium thiosulphate in the range of 0.027-0.033
(f) Mallow extract in the range of 0.9-1.1
(g) Rosemary oil in the range of 0.225-0.27
(h) Eucalyptus oil in the range of 0.225-0.27.

20. A method of treating skin ailments as claimed in claim 19 wherein said cream composition consisting of the following ingredients in the following range of proportions:
(a) Water in the range of 47.0205-27.4695
(b) Propylene glycol in the range of 3.6-4.4
(c) Mineral oil in the range of 2.7-3.3
(d) Cetyl alcohol in the range of 3.6-4.4
(e) Stearic acid in the range of 3.6-4.4
(f) Glycerol Tionostearate in the range of 2.7-3.3
(g) Coal tar topical solution in the range of 5.175-6.325
(h) Chamomile extract in the range of 0.9-1.1
(i) Methyl paraben in the range of. 0.225-0.275
(j) Propyl paraben in the range of 0.09-0.11
(k) Triethanolamine in the range of 0.54-0.66
(l) Milk amino acids in the range of 2.25-2.75
(m) Mallow extract in the range of 0.9-1.1
(n) Tea tree solution in the range of 2.7-3.3
(o) Dimethicone in the range of 0.45-0.55
(p) Carbomer in the range of 3.6-4.4
(q) Glycerine in the range of 2.7-3.3
(r) Petrolatum in the range of 0.9-1.1
(s) Cetearyl alcohol in the range of 2.25-2.75
(t) Ceteareth 20 in the range of 2.25-2.75
(u) Polysorbate 20 in the range of 1.8-2.2
(v) F D and C Yellow #5 solution in the range of 0.045-0.055
(w) D and C red #33 solution in the range of 0.0045-0.055.
